(19) United States
(12) Patent Application Publication
(10) Pub. No.: US 2024/0300949 A1
(43) Pub. Date: Sep. 12, 2024

(54) NOVEL CRYSTALLINE FORM OF 1-(8-BROMOPYRIDO[2,3-E][1,2,4]TRIAZOLO[4,3-A]PYRAZIN-4-YL)-N-METHYLAZETIDIN-3-AMINE HEMISUCCINATE

(71) Applicant: JW PHARMACEUTICAL CORPORATION, Gyeonggi-do (KR)

(72) Inventors: Kim Troensegaard NIELSEN, Ballerup (DK); Flemming Hofmann LARSEN, Ballerup (DK)

(21) Appl. No.: 18/573,510

(22) PCT Filed: Jun. 22, 2022
(86) PCT No.: PCT/EP2022/066945

§ 371 Date: Dec. 22, 2023

(30) Foreign Application Priority Data
Jun. 22, 2021 (EP) 21181009.8

(51) Int. Cl. C07D 471/14

(52) U.S. Cl. CPC C07D 471/14

(57) ABSTRACT

A novel crystalline form of 1-(8-bromopyrido[2,3-e][1,2,4]triazolo[4,3-a]pyrazin-4-yl)-N-methylazetidin-3-amine hemisuccinate is provided along with pharmaceutical compositions comprising the same. Also disclosed is the use of the novel polymorph for the treatment of diseases, such as atopic dermatitis (AD), itch, pruritus and various forms of urticaria for example chronic idiopathic urticaria subtypes.

### SUMMARY OF THE INVENTION

The present invention relates to a novel crystalline form of 1-(8-bromopyrido[2,3-e][1,2,4]triazolo[4,3-a]pyrazin-4-yl)-N-methylazetidin-3-amine hemisuccinate, pharmaceutical compositions comprising the novel crystalline form of 1-(8-bromopyrido[2,3-e][1,2,4]triazolo[4,3-a]pyrazin-4-yl)-N-methylazetidin-3-amine hemisuccinate and to the use of the novel crystalline form for the treatment of diseases, such as atopic dermatitis (AD), itch, pruritus and various forms of urticaria for example chronic idiopathic urticaria subtypes, such as cholinergic urticaria. Also provided herein is a method of preparing the crystalline form of the invention.

### BACKGROUND OF THE INVENTION

U.S. Pat. No. 9,586,959 relates, among other compounds, to the compound 1-(8-bromopyrido[2,3-e][1,2,4]triazolo[4,3-a]pyrazin-4-yl)-N-methylazetidin-3-amine and pharmaceutically acceptable salts thereof as well as pharmaceutical compositions comprising the same. The patent discloses the preparation of a number of salts of the compound 1-(8-bromopyrido[2,3-e][1,2,4]triazolo[4,3-a]pyrazin-4-yl)-N-methylazetidin-3-amine.

1-(8-bromopyrido[2,3-e][1,2,4]triazolo[4,3-a]pyrazin-4-yl)-N-methylazetidin-3-amine exhibits a strong histamine 4 receptor inhibitory effect and shows suppressive effects against histamine induced infiltration of inflammatory cells such as mast cells and eosinophils. The compound thus has strong anti-inflammatory and anti-itching effects and is therefore useful for treating a range of diseases such as those disclosed in U.S. Pat. No. 9,586,959, including AD.

Different crystalline solid forms of chemical compounds may have distinct physical properties such as e.g. chemical stability, physical stability, hygroscopicity, melting point, solubility, dissolution rate, morphology and bioavailability which make them more or less suitable as the selected active ingredient in a pharmaceutical product.

In addition, a chemical entity may exist in several different crystalline solid forms and these include different polymorphic forms that share the same sum formula (e.g. anhydrates) and different solvates (e.g. hemihydrate, monohydrate and dihydrates) of the same chemical entity which do not share the same sum formula. Such crystalline solid forms have distinct crystal structures and vary in physical properties. The different crystalline solid forms can be distinguished from each other by e.g. melting point, XRPD pattern, spectral characteristics (e.g. FT-IR, Raman and SS-NMR), and other physical and chemical properties. Chemical entities can also exist in amorphous form.

The actual crystalline form selected therefore plays an important role in the development and manufacture of an active pharmaceutical ingredient. Should a single crystal form be required, it is important that the crystallization process be robust and reliably produce the desired crystalline form in polymorphically pure form and that the crystalline form does not change (e.g. interconvert to a different crystalline form) during the relevant manufacturing processes, and/or during storage.

The novel crystalline form according to the invention is a hemisuccinate i.e. a salt form where there for each molecule of succinic acid is two molecules of 1-(8-bromopyrido[2,3-e][1,2,4]triazolo[4,3-a]pyrazin-4-yl)-N-methylazetidine in the crystal lattice.

A number of different salts of 1-(8-bromopyrido[2,3-e][1,2,4]triazolo[4,3-a]pyrazin-4-yl)-N-methylazetidine have been identified. Some of the salts appear as anhydrate, monohydrates and dihydrates each in several polymorphic forms that interconvert upon drying or loses water at relatively low temperature and they are therefore not suitable for development as a pharmaceutical.

One crystalline form of 1-(8-bromopyrido[2,3-e][1,2,4]triazolo[4,3-a]pyrazin-4-yl)-N-methylazetidin-3-amine hemisuccinate monohydrate has now been identified (hereinafter called form F).

Form F is a channel hydrate and generally channel hydrates/solvates are not very stable since the solvent molecules easily can move in and out of the channels and causes collapse of the crystal lattice. In the case of form F the water molecules are placed in the same molecular plane as the succinate molecules which stabilize the hydrate and makes form F particularly useful for use in a solid pharmaceutical product, such as in a tablet. Furthermore, form F can be dried under moderate drying conditions such as in a vacuum oven at 60° C. (see FIG. 8) without collapsing the crystal lattice. Also form F has been found suitable for large scale drying.

Further, crystal habit and the particle size distribution is quite similar from batch to batch of form F and the particle size distribution data is promising with regards to drug product processability.

Experiments have demonstrated that form F tolerates micronization without undergoing amorphization.

In addition, form F is a hemisuccinate salt which would allow for higher drug load. This can be very beneficial in case of high doses and to keep the tablets as small as possible for the best compliance for the patients.

### SUMMARY OF THE INVENTION

The present invention relates to crystalline 1-(8-bromopyrido[2,3-e][1,2,4]triazolo[4,3-a]pyrazin-4-yl)-N-methylazetidin-3-amine hemisuccinate monohydrate characterized by one or more XRPD reflections at approximately (°2θ) at about 20=8.6, 11.9 and/or 15.8 (+0.2 degrees).

The invention also relates to a pharmaceutical composition comprising the crystalline form mentioned above and a pharmaceutically acceptable carrier.

In one embodiment the invention relates to a compound or pharmaceutical composition as described above for the treatment of disease selected from atopic dermatitis, itch, pruritus, and various forms of urticaria, including chronic idiopathic urticaria subtypes.

### DETAILED DESCRIPTION OF THE INVENTION

The technical problem underlying the present invention is to circumvent the drawbacks of other crystalline and/or amorphous forms of 1-(8-bromopyrido[2,3-e][1,2,4]triazolo[4,3-a]pyrazin-4-yl)-N-methylazetidin-3-amine such as, ability to form crystals, filtration properties, solubility, thermodynamic properties, stability issues (e.g. due to water uptake), density, and transformation (e.g. interconversion to other polymorphic forms or hydrates/anhydrates) at varying degrees of humidity and during crystallization processes.

### Definitions

As used herein the term “rt” or “room temperature” indicates that the applied temperature is not critical and that no exact temperature value have to be kept. Usually, “rt” or “room temperature” is understood to mean temperatures of about 15° C. to about 25° C. [see e.g. EU Pharmacopoeia 7.5, 1.2 (2012)].

The term “solvate” as used herein describes a crystalline compound in which solvent molecules are incorporated into the crystal lattice of the compound in a stoichiometric or non-stoichiometric manner. If the solvent molecules are water the term “hydrate” is used herein.

The type of hydrate depends on the molar ratio of water molecules to 1-(8-bromopyrido[2,3-e][1,2,4]triazolo[4,3-a]pyrazin-4-yl)-N-methylazetidin-3-amine hemisuccinate molecules.

The term “monohydrate” implies 0.8 to 1.2 mol water per mol of 1-(8-bromopyrido[2,3-e][1,2,4]triazolo[4,3-a]pyrazin-4-yl)-N-methylazetidin-3-amine hemisuccinate.

The term “non-hygroscopic” as used herein indicates that the increase in mass of a drug substance between about 0% to 80% relative humidity is less than 0.2% by weight.

In the context of the present invention, the term “XRPD reflection peak” denotes a particular 20 position in an XRPD pattern, wherein the signal-to-noise ratio (calculated according to item 2.2.46 of the European Pharmacopoeia) is greater than 3/1. “Absence of a peak” is herein defined as a peak having an intensity of at most 1%, such as 0.5% or 0.2%, of the highest peak in an XRPD of a sample of the compound of the invention, i.e no detectable XRPD peak above background signals.

In an XRPD pattern, the main characteristics of diffraction line profiles are 20 position, peak height, peak area and shape (characterized by, for example, peak width or asymmetry, analytical function, empirical representation). The 20 position is the most important factor as for example the intensity will be affected by sample preparation, and the width of the peaks by particle size. In addition to the diffraction peaks, an X-ray diffraction experiment also generates a more-or-less uniform background in an XRPD pattern, upon which the peaks are superimposed. Besides specimen preparation, other factors contribute to the background, for instance the sample holder, diffuse scattering from air and equipment, other instrumental parameters such as detector noise, general radiation from the X-ray tube, etc. The peak-to-background ratio can be increased by minimizing background and/or by choosing prolonged exposure times.

### ABBREVIATIONS

DSC: Differential Scanning Calorimetry
DVS: Dynamic Vapor Sorption
TGA: Thermogravimetric Analysis
XRPD: X-ray Powder Diffraction
13C CP/MAS NMR: ¹³C cross polarization magic angle spinning nuclear magnetic resonance
SXRD: Single crystal X-ray Diffraction

### BRIEF DESCRIPTION OF THE DRAWINGS

FIG. 1: XRPD pattern for form F (3-60° 2theta)

FIG. 2: XRPD pattern for form F (3-30° 2theta)

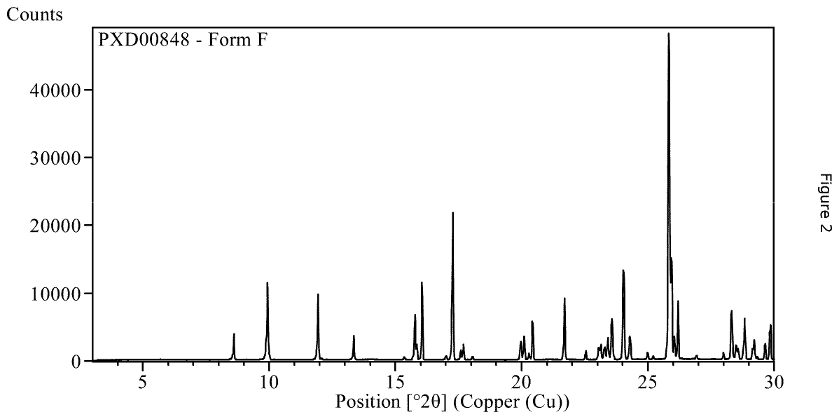

FIG. 3: DSC and TGA curve for form F

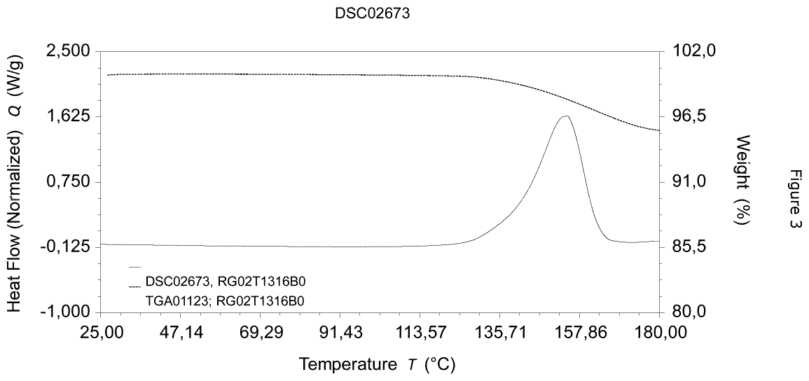

FIG. 4: The ORTREP drawing of the absolute crystal structure of form F. The hydrogen atoms have been excluded for an easier view.

FIG. 5: ¹³C CP/MAS NMR spectrum of Form F. The integrals of the aliphatic carbons confirm that this is a hemisuccinate salt as the integrals of the aliphatic carbons adds up to 5.18 in good agreement with four carbons from the side chain of the molecule and one carbon from one CH₂in a succinic acid molecule.

FIG. 6: TGA of Form F run for 24 hours at constant temperature of 60° C.

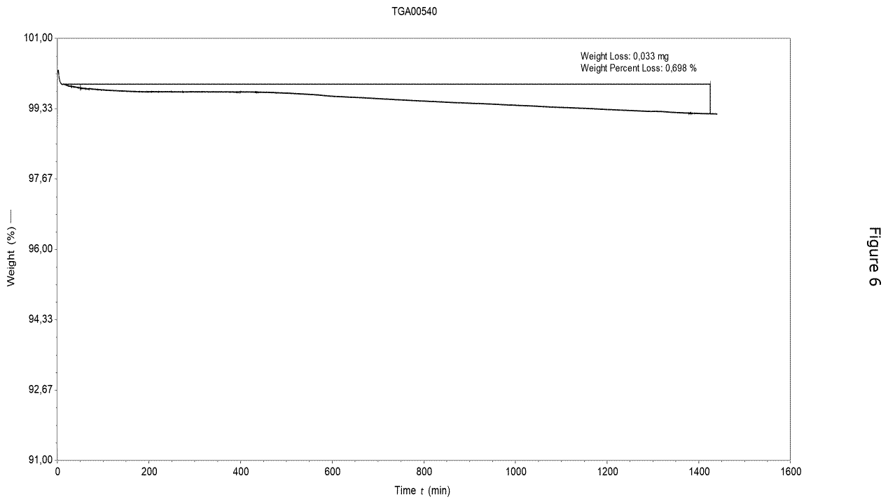

FIG. 7: XRPD time series (0-8 hours) of Form F.

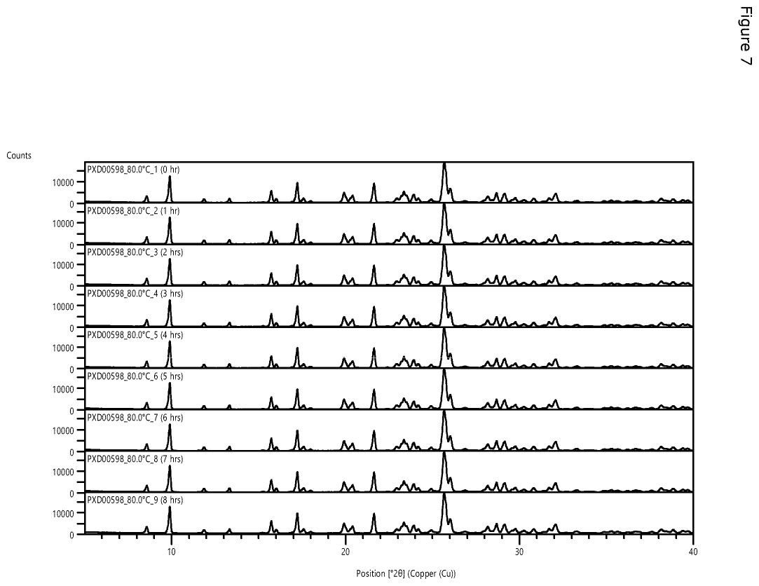

FIG. 8: XRPD's of Form F stored in vacuum at 60° for different periods of time. At each time point of a quick XRPD (9 min) and a normal XRPD (1 hour) were recorded

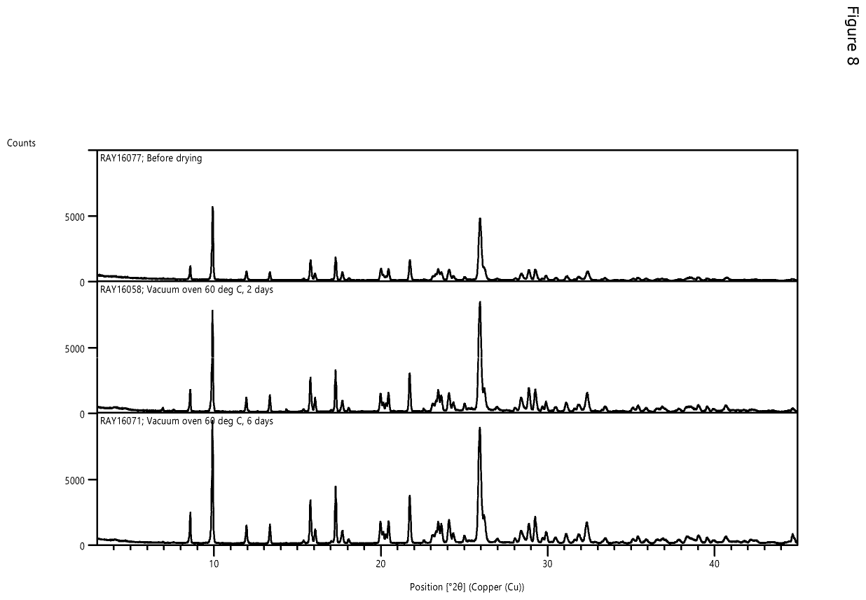

FIG. 9: XRPD of Form F prepared as powder (upper diffractogram), tablet prepared at a pressure of 5.5(middle diffractogram) and tablet prepared at a pressure of 7.0 (lower diffratogram)

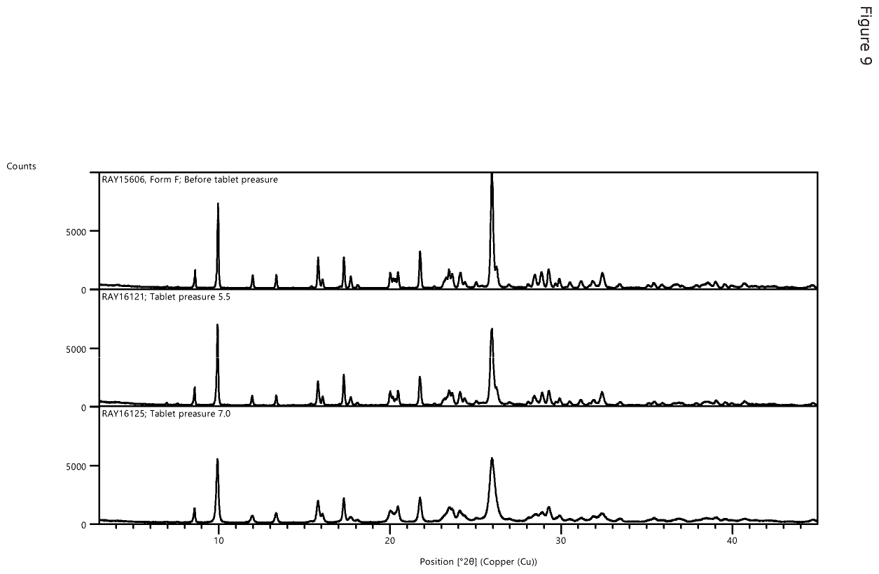

FIG. 10: Single crystal structure of—form F shown along ^{A)}a-axis, ^{B)}b-axis, ^{C)}c-axis and ^{D)}molecular plane. Carbon atoms are displayed in gray, nitrogen atoms in blue, oxygen atoms in red and bromine atoms in orange. Along the b-axis it is seen that base ring systems are stacked in opposite directions and along the molecular plane it is seen that succinic acid and water are located in planes in between the base molecules.

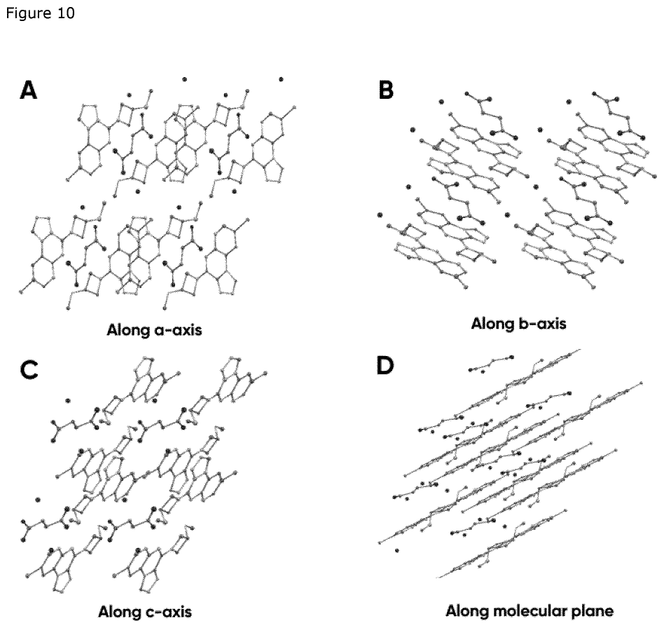

### TABLE 1 BELOW SHOW THE SXRD OF FORM F

### Single Crystal X-Ray Analysis

TABLE 1
The crystal parameters from the single
crystal structure determination

LEO152020G, form F

Crystal data

| | |
|---|---|
| Chemical formula | 2(C₁₂H₁₂BrN₇)•C₄H₆O₄•2(H₂O) |
| Mᵣ | 822.47 |
| Crystal system, | Triclinic, P1 |
| space group | |
| Temperature (K) | 120 |
| a, b, c (Å) | 7.8767 (2), 9.8582 (3), 11.7062 (3) |
| a, b, g (°) | 110.731 (2), 106.260 (2), 98.961 (2) |
| V (Å³) | 782.55 (4) |
| Z | 1 |
| Radiation type | Cu Ka |
| m (mm⁻¹) | 3.88 |

Data collection

| | |
|---|---|
| Diffractometer | SuperNova, Dual, Cu at zero, Atlas |
| | diffractometer |
| Absorption | Multi-scan |
| correction | CrysAlis PRO, Agilent Technologies, |
| | Version 1.171.36.28 (release Jan. 2, 2013 |
| | CrysAlis171 .NET) (compiled Feb. 1 |
| | 2013, 16:14:44) Empirical absorption |
| | correction using spherical harmonics, |
| | implemented in SCALE3 ABSPACK |
| | scaling algorithm. |
| No. of measured, | 11730, 5706, 5692 |
| independent and | |
| observed [I³ | |
| 2 u(I)] reflections | |
| Rᵢₙₜ | 0.021 |
| (sin q/l)ₘₐₓ (Å⁻¹) | 0.623 |

Refinement

| | |
|---|---|
| R[F² > 2 s(F²)], | 0.048, 0.177, 1.69 |
| wR(F²), S | |
| No. of reflections | 5706 |
| No. of parameters | 211 |
| No. of restraints | 3 |
| (D/s)ₘₐₓ | 1.663 |
| Dρₘₐₓ, Dρₘᵢₙ | 1.01, −0.77 |
| (e Å⁻³) | |

In one embodiment the invention relates to acrystalline form of 1-(8-bromopyrido[2,3-e][1,2,4]triazolo[4,3-a]pyrazin-4-yl)-N-methylazetidin-3-amine hemisuccinate.

In a further embodiment the invention relates to a crystalline compound as defined above, wherein the XRPD reflections comprise one or more XRPD reflections at approximately (°2θ) 8.6, 11.9, 15.8 and/or 25.8 (±0.2 degrees).

In one further embodiment the invention relates to a crystalline compound as defined above wherein the XRPD reflections comprise one or more XRPD reflections at approximately (°2θ) 8.6, 9.9, 11.9, 13.3. 15.8, 16.1, 17.3 and/or 21.7 (±0.2 degrees).

In a further embodiment the invention relates to a crystalline compound as defined above, wherein the XRPD reflections comprise one or more XRPD reflections at approximately 8.6, 11.9 and 15.8 (±0.2 degrees).

In a further embodiment the invention relates to a crystalline compound as defined above wherein the XRPD reflections comprise XRPD reflections at approximately (°2θ) 8.6, 11.9, 15.8 and 25.8 (±0.2 degrees).

In a further embodiment the invention relates to a crystalline compound as defined above wherein the XRPD reflections comprise XRPD reflections at approximately (°2θ) 8.6, 9.9, 11.9, 13.3. 15.8, 16.1, 17.3 and 21.7(±0.2 degrees).

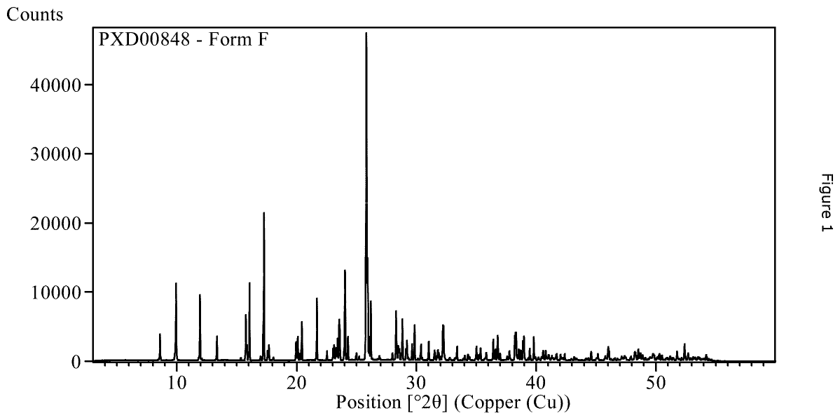

In a further embodiment the invention relates to a crystalline compound as defined above wherein the crystalline compound has an XRPD pattern essentially similar to the XRPD pattern in FIG. 1.

In a further embodiment the invention relates to a crystalline compound as defined above, wherein the crystalline compound has an XRPD pattern according to the XRPD pattern in FIG. 1.

In a further embodiment the invention relates to a crystalline compound as defined above, wherein the crystalline compound is characterized by a solid state ¹³C CP/MAS NMR spectrum with peaks at one or more of 180, 60.0, 50.3 and/or 34.2 ppm±0.2 ppm.

In a further embodiment the invention relates to a crystalline compound as defined above, wherein the crystalline compound is characterized by a solid state ¹³C CP/MAS NMR spectrum with peaks at one or more of 180, 146.7, 140.5, 138.1, 130.1, 118.2, 60.0, 56.8, 50.3 and/or 34.2 ppm±0.2 ppm.

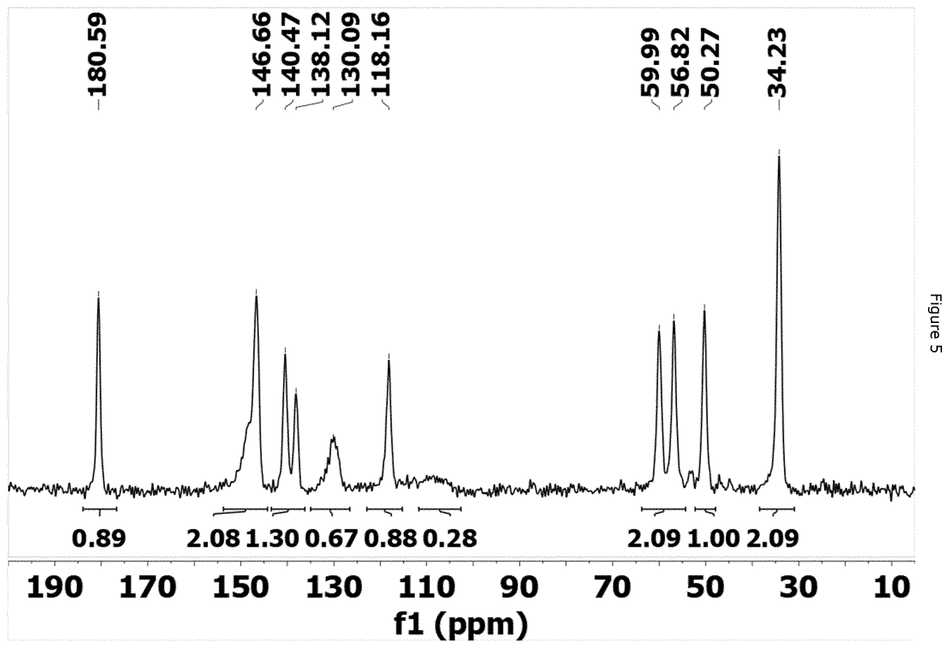

In a further embodiment the invention relates to a crystalline compound as defined above characterized by having a ¹³C CP/MAS NMR spectrum essentially similar to the ¹³C CP/MAS NMR spectrum in FIG. 5.

In a further embodiment the invention relates to a crystalline compound as defined above by having a ¹³C CP/MAS NMR spectrum according to the ¹³C CP/MAS NMR spectrum in FIG. 5.

In a further embodiment the invention relates to a crystalline compound having a ¹³C CP/MAS NMR spectrum as defined above, further characterized by one or more XRPD reflections at approximately (°2θ) 8.6, 9.9, 11.9, 13.3. 15.8, 16.1, 17.3 and 21.7 (±0.2 degrees).

In a further embodiment the invention relates to a crystalline compound as above which has a DSC curve comprising an endothermic event with a onset value at about 138.4±2° C.

In a further embodiment of the invention Form F is characterized by having single crystal parameters which are substantially the same as those provided in Table 1.

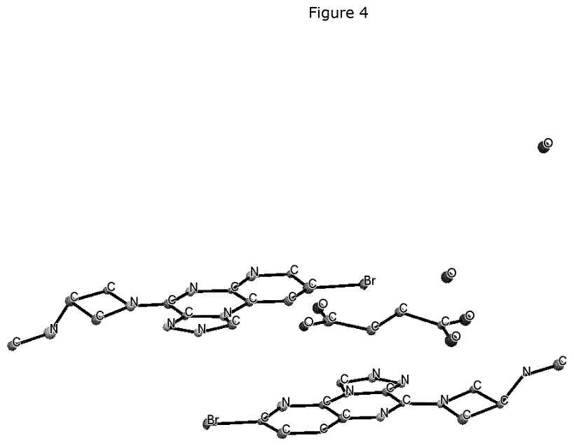

In a more particularly preferred embodiment of the present invention, the form F has a structure obtained by single-crystal X-Ray crystallography (SXRC) as shown in FIG. 4.

In a further embodiment the invention relates to a crystalline compound as above, wherein the molar ratio of 1-(8-bromopyrido[2,3-e][1,2,4]triazolo[4,3-a]pyrazin-4-yl)-N-methylazetidin-3-amine to succinic acid is in the range of from 2:1.2 to 2:0.8, and preferably is approximately 2:1

In a further embodiment the invention relates to a crystalline compound, wherein the molar ratio of 1-(8-bromopyrido[2,3-e][1,2,4]triazolo[4,3-a]pyrazin-4-yl)-N-methylazetidin-3-amine hemisuccinate to water is in the range from 1:0.8 to 1:1.2, and preferably is approximately 1:1.

### Method of Preparation of Form F

The crystalline form of the invention may be prepared from 1-(8-bromopyrido[2,3-e][1,2,4]triazolo[4,3-a]pyrazin-4-yl)-N-methylazetidin-3-amine in the form of the free base by crystallization from a suitable solvent, such as a lower alcohol (such as methanol, ethanol, propanol and mixtures thereof), acetone, acetonitrile, lower alkyl acetate (such as ethyl acetate and propyl acetate), tetrahydrofuran and mixtures of these solvents with various amounts of water, and/or liquid hydrocarbons (such as hexane and heptane) at rt or cooled to temperatures below rt such as temperatures below 0° C., suitably−18° C.

In one specific embodiment the solvent is 1-propanol, and the crystallization is carried out at rt or above rt or lower temperatures such as −18° C.

The crystalline form of the invention is formed by heating the free base in a suitable solvent in the presence of the required amount of succinic acid, addition of water followed by cooling of the reaction mixture.

The amount of succinic acid is suitably 0.5-0.6 or more suitably 0.55 equivalents to 1 equivalent of 1-(8-bromopyrido[2,3-e][1,2,4]triazolo[4,3-a]pyrazin-4-yl)-N-methylazetidin-3-amine.

The 1-(8-bromopyrido[2,3-e][1,2,4]triazolo[4,3-a]pyrazin-4-yl)-N-methylazetidin-3-amine is suitably suspended in the solvent by heating of the reaction mixture to 40° C., and once most of the 1-(8-bromopyrido[2,3-e][1,2,4]triazolo[4,3-a]pyrazin-4-yl)-N-methylazetidin-3-amine is dissolved, a small amount of water is added. The amount of water to the amount of solvent is suitably in the range 1:15 to 1:25, suitably 1:20.

Crystalline form F of the invention have advantages over other salt forms as milder reaction conditions, i.e around 0.5 equivalent of the weak acid succinic acid is used as compared to salt forms prepared from stronger acids in much larger amounts e.g. used to prepare e.g. a sulfate salt of 1-(8-bromopyrido[2,3-e][1,2,4]triazolo[4,3-a]pyrazin-4-yl)-N-methylazetidin-3-amine.

Because of the mild reaction conditions there is essentially no degradation of the 1-(8-bromopyrido[2,3-e][1,2,4]triazolo[4,3-a]pyrazin-4-yl)-N-methylazetidin-3-amine molecule during the final salt formation, and there is a significantly lower impurity burden leading to a higher purity product mainly as a result of reduced degradation.

A further aspect of the present invention is directed to a pharmaceutical composition comprising the crystalline compound of the present invention and at least one pharmaceutically acceptable excipient. The pharmaceutical composition may be an oral dosage form, preferably a tablet and/or capsule.

In addition, the present invention relates to the use of the crystalline compound of the present invention for the preparation of a solid medicament.

In another embodiment the present invention relates to solid pharmaceutical compositions comprising an effective amount of the crystalline compound of the present invention and a pharmaceutically acceptable carrier as well as to processes of preparing the same. Moreover, the present invention is directed to the pharmaceutical composition of the present invention and/or the crystalline compound of the present invention for use in the treatment of any of the disease or disorders mentioned in U.S. Pat. No. 9,586,959, including diseases and disorders such as atopic dermatitis (AD), itch, pruritus and any of the various types of urticaria.

The pharmaceutical compositions of the present invention comprising the crystalline compound of the present invention may further comprise one or more pharmaceutically acceptable excipients. Such excipients are preferably selected from the group consisting of diluents, sweeteners, buffering agents, glidants, flowing agents, flavouring agents, lubricants, preservatives, surfactants, wetting agents, binders, disintegrants and thickeners. Other excipients known in the field of pharmaceutical compositions may also be used. Furthermore, the pharmaceutical composition may comprise a combination of two or more excipients also within one of the members of the above-mentioned group.

Suitable binders which can be used for the pharmaceutical compositions of the present invention comprising the crystalline compound of the present invention, further comprise e.g. alkylcelluloses such as methylcellulose, hydroxyalkylcelluloses such as hydroxymethylcellulose, hydroxyethylcellulose, hydroxypropylcellulose and hydroxybutylcellulose, hydroxyalkylalkylcelluloses such as hydroxyethylmethylcellulose and hydroxypropylmethylcellulose, carboxyalkylcelluoses such as carboxymethylcellulose, alkali metal salts of carboxyalkylcelluloses such as sodium carboxymethylcellulose, carboxyalkylalkylcelluloses such as carboxymethylethylcellulose, carboxyalkylcellulose esters, starches such as starch 1551, modified starches such as sodium carboxymethyl starch, pectins, chitin derivatives such as chitosan, heparin and heparinoids, polysaccharides such as alginic acid, alkali metal and ammonium salts thereof, carrageenans, galactomannans, tragacanth, agar-agar, gum arabic, guar gum and xanthan gum, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, polyalkylene oxides such as polyethylene oxide and polypropylene oxide and copolymers of ethylene oxide and propylene oxide, e.g. poloxamers and poloxamines, copovidone.

Suitable diluents which can be used for the pharmaceutical compositions of the present invention comprising the crystalline compound of the present invention further comprise e.g. calcium carbonate, dibasic calcium phosphate, dibasic calcium phosphate dihydrate, tribasic calcium phosphate, calcium sulphate, microcrystalline cellulose including silicified microcrystalline cellulose, powdered cellulose, dextrates, dextrin, dextrose excipient, fructose, kaolin, lactitol, lactose anhydrous, lactose monohydrate, mannitol, sorbitol, starch, modified starch, sodium chloride, sucrose, compressible sugar, confectioner's sugar, a spray-dried mixture of lactose monohydrate and microcrystalline cellulose (75:25), commercially available as Microcelac®, a co-processed spray-dried mixture of microcrystalline cellulose and colloidal silicon dioxide (98:2), commercially available as Prosolv®.

Suitable glidants which can be used for the pharmaceutical compositions of the present invention comprising the crystalline compound of the present invention further comprise e.g. talc, colloidal silicon dioxide, starch and magnesium stearate.

Suitable disintegrants which can be used for the pharmaceutical compositions of the present invention comprising the crystalline compound of the present invention further comprise e.g. starch, ion exchange resins, e.g. Amberlite, cross-linked polyvinylpyrrolidone, modified cellulose gum, e.g croscarmellose sodium, sodium starch glycolate, sodium carboxymethylcellulose, sodium dodecyl sulphate, modified corn starch, microcrystalline cellulose, magnesium aluminium silicate, alginic acid, alginate and powdered cellulose.

Suitable lubricants which can also be used for the pharmaceutical compositions of the present invention comprising the crystalline compound of the present invention further comprise e.g. magnesium stearate, calcium stearate, stearic acid, talc, polyethylene glycol, sodium lauryl sulphate and magnesium lauryl sulphate.

Some formulations, e.g. tablets may contain ingredients that have XRPD reflection peaks in the same position or area as the crystalline compound of the invention or have broad peaks. These may hide some of the XRPD pattern or peaks of the crystalline compound of the invention when the XRPD experiment is performed on a formulation comprising the crystalline compound of the invention as opposed to the pure crystalline salt alone. This means that one cannot always see all XRPD reflection peaks of the crystalline compound of the invention when an XRPD experiment is performed on a formulation of the crystalline compound

Thus, according to one embodiment, the invention relates to a pharmaceutical composition comprising a crystalline compound as defined herein together with pharmaceutically acceptable vehicle, excipient or pharmaceutically acceptable carrier(s), wherein said pharmaceutically acceptable vehicle, excipient or pharmaceutically acceptable carrier(s) comprises one or more ingredients which exhibit XRPD reflection peaks including one or more XRPD reflection peaks that overlap with and hide one or more XRPD reflection peaks of the crystalline compound of the invention.

The same issues may arise with solid state NMR where for example intense signals from a cellulose component should be expected in the spectral region 60-110 ppm and the peaks from stearate will be seen in the spectral region 15-40 ppm—along with a carbonyl peak around 172 ppm.

Thus, according to one embodiment, the invention relates to a pharmaceutical composition comprising a crystalline compound as defined herein together with pharmaceutically acceptable vehicle, excipient or pharmaceutically acceptable carrier(s), wherein said pharmaceutically acceptable vehicle, excipient or pharmaceutically acceptable carrier(s) comprises one or more ingredients which are characterized by a ¹³C CP/MAS NMR spectrum that may include one or more peaks that overlap with and hide one or more ¹³C CP/MAS NMR peaks of the crystalline compound of the invention.

The absence of other crystalline forms of 1-(8-bromopyrido[2,3-e][1,2,4]triazolo[4,3-a]pyrazin-4-yl)-N-methylazetidin-3-amine can be tested by comparing an XRPD pattern taken of any crystalline form of 1-(8-bromopyrido[2,3-e][1,2,4]triazolo[4,3-a]pyrazin-4-yl)-N-methylazetidin-3-amine or a salt thereof with an XRPD pattern of form F as obtained e.g. from example 1 and shown in FIG. 1. For such a comparison, the XRPD pattern shown in FIG. 1 can be taken as an XRPD pattern of 100% pure crystalline compound of form F of the present invention.

### Description of the Test Methods Used to Characterize the Polymorphic Forms Disclosed Herein

### XRPD:

XRPD patterns were collected with a PANalytical X'pert Empyrean diffractometer using an incident Cu Kα radiation and operating at 45 kV and 40 mA. The XRPD patterns were collected in the 2 theta range from 3 to 60 degrees with a step size of 0.013°, counting time of 198.645s and in transmission geometry. 25 repetitions were done and summarized. In the incident beam path, an parabolic shaped beam Cu W/Si (hybrid MPD) monochromator together with a 10 mm fixed mask, fixed anti-scatter slit ⅛° and fixed divergence slits of 1/16° were placed to line focus the Cu Kα X-rays through the sample and onto the detector. At the diffracted beam path, a long anti-scatter extension was placed to minimize the background generated by air. Furthermore, Soller slits of 0.02 rad were placed on both the incident and diffracted beam paths to minimize broadening from axial divergence.

The intensities measured in XRPD may vary considerably between samples of the same crystal structure due to orientation in the sample (orientation effects). The intensities measured in XRPD will also include experimental error. Measured peak intensities will vary depending on various experimental factors such as the equipment used, the testing conditions employed, the size of the sample, the crystallinity of the material (the degree of structural order) and the sample preparation.

The sample was placed on a 3 μm thick foil rotated with a speed of 16 s for one turn for better particle statistics. The diffraction patterns were collected using a PIXel RTMS detector with an active length of 3.347° and located 240 mm from the sample.

### SS-NMR

Solid-state ¹³C cross polarization (CP) magic angle spinning (MAS) NMR spectra were recorded using a Bruker Avance III HD 600 NMR instrument operating at Larmor frequencies of 150.9 and 600.13 MHz for ¹³C and ¹H, respectively. The experiments were conducted using a double tuned CP/MAS probe equipped for 4 mm (outer diameter, o.d.) spinners. All samples were packed in 4 mm (o.d.) zirconia spinners. The CP/MAS NMR spectra were recorded using with variable amplitude cross polarization and high-power proton decoupling (TPPM) during acquisition. The operation conditions were: temperature: 298 K; contact time: 6 ms; recycle delay: 16 s, 128 scans; spin rate of 14.1 kHz. Chemical shifts were referenced to an external sample of α-glycine (carbonyl carbon chemical shift assigned to 176.5 ppm relative to the signal of tetramethylsilane).

### TGA

### Thermo Gravimetric Analysis (TGA)

TGA experiments were conducted using a TGA550 instrument from TA Instruments. About 1-10 mg of sample was loaded into a ceramic pan for the measurements. The sample temperature was ramped from 25 to 500° C. at 10° C./min. Nitrogen was used as the purge gas at a flow rate of 50 mL/min.

### DSC

### Differential Scanning Calorimetry (DSC)

DSC: Heating rate of 10° C./min under a nitrogen atmosphere. About 1-2 mg of sample was loaded into an open aluminum pan for the measurements. Instrument Q20 from TA Instruments.

### DVS:

### Instrument: DVS Advantage

Methods: About 5 mg of the substance was added into Al pan and exposed to stepwise RH changes during two consecutive cycles according to; 20-30-40-50-60-70-80-70-60-50-40-30-20-10-0-10-20-30-40-50-60-70-80-90-80-70-60-50-40-30-20-10-0% RH using open loop mode. The experiments were performed using a gas flow rate of 200 ml/min and at 25° C. The dm/dt criteria applied was 0.001 weight-%/min during a 5 minutes window, with a maximum allowed time of 150 minutes for all steps, except for the steps at 0% RH which had no criteria but were set to 6 h.

### Single-Crystal X-Ray Diffraction

Data were collected using a SuperNova, Dual diffractometer with an Atlas CCD area detector (Temperature: 120(2) K; Cu Kα Radiation λ=1.5418 Å; data collection method: ω scans). Further details can be found in the table above. Program(s) used to solve structure: CrysAlisPro, Agilent Technologies, Version 1.171.37.34 (release 22-05-2014 CrysAlis171 .NET), ShelXL (Sheldrick, 2008) used to refine structure and Olex2 (Dolomanov et al., 2009) for ORTEP drawings.

The given error ranges in this application for the spectroscopic characteristics, including those in the claims, may be more or less depending on factors well known to a person skilled in the art of spectroscopy and may for example depend on sample preparation, such as particle size distribution, or if the crystal form is part of a formulation, on the composition of the formulation, as well as instrumental fluctuations, and other factors.

In the following the present invention will be described in further detail by illustrative, non-limiting examples.

### EXAMPLES

### Example 1: Preparation of 1-(8-bromopyrido[2,3-e][1,2,4]triazolo[4,3-a]pyrazin-4-yl)-N-methylazetidin-3-amine hemisuccinate monohydrate

1-(8-Bromopyrido[2,3-e][1,2,4]triazolo[4,3-a]pyrazin-4-yl)-N-methylazetidin-3-amine (1.0 equiv.) and succinic acid (0.55 equiv.) were mixed. 1-Propanol (40 mL/g) was added. The reaction mixture was heated to 40° C. and stirred under an inert atmosphere. Water (2 mL/g) was added, and the reaction mixture was stirred at 40° C. for 30 minutes before it was cooled to 20° C. over a period of 60 minutes. The resulting mixture was stirred at 20° C. for 18 hours. The reaction mixture was filtered, and the filter cake was washed with 1-propanol (5 mL/g) and dried under vacuum at 50° C. ¹H NMR of the isolated crystalline compound confirmed the presence of a hemisuccinate salt (¹H NMR (600 MHz): 9.95 ppm (s, 1H), 8.89 ppm (d, J=2.3 Hz, 1H), 8.58 ppm (d, J=2.3 Hz, 1H), 4.78-5.01 ppm (m, 1H), 4.31-4.55 ppm (m, 2H), 3.94-4.10 ppm (m, 1H), 3.76 ppm (tt, J=4.9, 7.2 Hz, 1H), 2.33 ppm (s, 3H), 2.39 ppm (s, 2H, succinate)). The fact that the integral of the succinate only amounts to a relative amount of two hydrogens confirm that the molar ratio is 1.0:0.5.

The XRPD of the crystalline compound is shown inf FIG. 1.

1. Crystalline 1-(8-bromopyrido[2,3-e][1,2,4]triazolo[4,3-a]pyrazin-4-yl)-N-methylazetidin-3-amine hemisuccinate monohydrate characterized by one or more XRPD reflections at approximately (°2θ) at about 2θ=8.6, 11.9 and/or 15.8 (±0.2 degrees).

2. The crystalline compound according to claim 1, characterized by one or more XRPD reflections at approximately (°2θ) 8.6, 11.9, 15.8 and/or 25.8 (±0.2 degrees).

3. The crystalline compound according to claim 1, characterized by one or more XRPD reflections at approximately (°2θ) 8.6, 9.9, 11.9, 13.3. 15.8, 16.1, 17.3 and/or 21.7 (±0.2 degrees).

4. The crystalline compound according to claim 1, characterized by one or more XRPD reflections at approximately 8.6, 11.9 and 15.8 (±0.2 degrees).

5. The crystalline compound according to claim 1, characterized by one or more XRPD reflections at approximately (°2θ) 8.6, 11.9, 15.8 and 25.8 (±0.2 degrees).

6. The crystalline compound according to claim 1, characterized by one or more XRPD reflections at approximately (°2θ) 8.6, 9.9, 11.9, 13.3. 15.8, 16.1, 17.3 and 21.7 (±0.2 degrees).

7. The crystalline compound according to claim 1, wherein the crystalline compound has an XRPD pattern essentially similar to the XRPD pattern in FIG. 1.

8. The crystalline compound according to claim 1, wherein the crystalline compound has an XRPD pattern according to the XRPD pattern in FIG. 1.

9. The crystalline compound according to claim 1, wherein the crystalline compound is characterized by a solid state ¹³C CP/MAS NMR spectrum with peaks at one or more of 180, 60.0, 50.3 and/or 34.2 ppm±0.2 ppm.

10. The crystalline compound according to claim 1, wherein the crystalline compound is characterized by a solid state ¹³C CP/MAS NMR spectrum with peaks at one or more of 180, 146.7, 140.5, 138.1, 130.1, 118.2, 60.0, 56.8, 50.3 and/or 34.2 ppm±0.2 ppm.

11. The crystalline compound according to claim 9 characterized by having a ¹³C CP/MAS NMR spectrum essentially similar to the ¹³C CP/MAS NMR spectrum in FIG. 5.

12. The crystalline compound according to claim 9 characterized by having a ¹³C CP/MAS NMR spectrum according to the ¹³C CP/MAS NMR spectrum in FIG. 5.

13. The crystalline compound according to claim 9, further characterized by one or more XRPD reflections at approximately (°2θ) 8.6, 9.9, 11.9, 13.3. 15.8, 16.1, 17.3 and 21.7 (±0.2 degrees).

14. The crystalline compound according to claim 1, wherein the crystalline compound is characterized by having the single-crystal X-Ray crystallography (SXRC) parameters as shown in table 1

15. The crystalline salt according to claim 1 which has a DSC curve comprising an endothermic event with an onset value at about 138.4±2° C.

16. The crystalline compound of claim 1, characterized in that the molar ratio of 1-(8-bromopyrido[2,3-e][1,2,4]triazolo[4,3-a]pyrazin-4-yl)-N-methylazetidin-3-amine to succinic acid is in the range of from 2:1.2 to 2:0.8, and preferably is approximately 2:1

17. The crystalline compound according to claim 1, wherein the molar ratio of 1-(8-bromopyrido[2,3-e][1,2,4]triazolo[4,3-a]pyrazin-4-yl)-N-methylazetidin-3-amine hemisuccinate to water is in the range from 1:0.8 to 1:1.2, and preferably is approximately 1:1.

18. A pharmaceutical composition comprising the crystalline salt according to claim 1 and a pharmaceutically acceptable carrier.

19. A method for treatment of disease selected from atopic dermatitis, itch, pruritus, and various forms of urticaria, comprising:
administering to a subject in need thereof a therapeutically effective amount of the crystalline compound of claim 1.

20. The method according to claim 19, wherein the forms of urticaria include chronic idiopathic urticaria subtypes.

21. The method according to claim 20, wherein the chronic idiopathic urticaria subtypes include cholinergic urticaria.
